(19) United States
(12) Patent Application Publication
(10) Pub. No.: US 2026/0007374 A1
(43) Pub. Date: Jan. 8, 2026

(54) DYNAMIC MYELOGRAPHY POSITIONING DEVICE

(71) Applicant: The Regents of the University of Colorado, a Body Corporate, Denver, CO (US)

(72) Inventors: Andrew L. Callen, Denver, CO (US); Michael Bojanowski, Denver, CO (US); Joshua Thomas Grip, Arvada, CO (US)

(21) Appl. No.: 19/330,927

(22) Filed: Sep. 17, 2025

Related U.S. Application Data
(63) Continuation of application No. PCT/US2024/021088, filed on Mar. 22, 2024.
(60) Provisional application No. 63/453,791, filed on Mar. 22, 2023.

(51) Int. Cl. A61B 6/04; A61B 5/03; A61B 6/00

(52) U.S. Cl. CPC A61B 6/0487; A61B 5/032; A61B 6/0442; A61B 6/481

(57) ABSTRACT

A method of performing dynamic myelography and a dynamic myelography positioning device configured to alter the orientation of a patient easily and quickly. The device is configured to move a patient from a generally flat laying position into a sloped or partially sloped position. In some embodiments, the device is attachable or configured to rest upon an existing imaging table. The device includes a frame with a patient supporting structure integrated with or attached to the frame. A pivot mechanism having a rotational axis enables the two ends of the frame to fold towards each other. An actuator is in operable communication with the frame to control the degree of rotation of the ends of the frame about the rotational axis of the pivot mechanism.

### CROSS-REFERENCE TO RELATED APPLICATIONS

This nonprovisional application is a continuation of PCT International Patent Application No. PCT/US2024/021088 entitled "Dynamic Myelography Positioning Device" with an international filing date of March 22, 2024, by the same inventors, which claims priority to US provisional application No. 63/453,791, entitled "Dynamic Myelography Positioning Device," filed March 22, 2023, by the same inventors.

### BACKGROUND OF THE INVENTION

### 1. Field of the Invention

This invention relates, generally, to medical devices. More specifically, it relates to medical devices to aid in medical imaging.

### 2. Brief Description of the Prior Art

Spontaneous intracranial hypotension (SIH) is a complex clinical syndrome resulting from abnormal leakage of cerebrospinal fluid (CSF) from the spine, resulting in a debilitating headache disorder. Its diagnosis often requires a complex synthesis of clinical symptoms and imaging findings, and in order to localize a CSF leak, an advanced procedure referred to as dynamic myelography must be performed, using either computed tomographic (CT) or digital subtraction (DSM) techniques. Our understanding of SIH and CSF leaks is in its relative infancy; in fact, one of the major contributing pathologies to SIH - CSF-venous fistula - was not discovered until 2014. Thus, the ability to perform dynamic CTM or DSM to localize and eventually treat these CSF leaks is limited to very few institutions. Additionally, we have come to learn that conventional MR imaging of the brain, which was traditionally conceptualized as highly sensitive for the presence of an underlying CSF leak, can be falsely negative in at least 19% of individuals. Therefore, in order to effectively care for patients potentially suffering from SIH, an institution must have not only the technical expertise to perform these advanced procedures, but also the clinical acumen to suspect SIH in patients in whom "conventional" first-line imaging does not demonstrate findings suggestive of a CSF leak, and refer them for advanced diagnostic testing and treatment.

Currently, in order to definitively diagnose a CSF leak or fistula, dynamic myelography must be performed. Myelography refers to a procedure whereby x-ray dye is injected into the CSF and subsequently visualized with x-ray or CT imaging. What distinguishes "dynamic" myelography from conventional myelography is that because certain types of subtle leaks (namely CVF) are transient, images must be obtained immediately after injection of contrast material, and provocative maneuvers are performed such as patient elevation, CSF pressure augmentation, and inspiration in order to increase visual conspicuity of the leak. As dynamic myelography is relatively new, it is executed in different ways depending on the institution at which it is performed. Some institutions place foam wedge 10 under the hips of patient 12, as shown in FIG. 1, so that as contrast is injected into the lower part of the spine, it immediately moves towards the top of the spine. While often successful, foam wedge 10 is not adjustable in height to account for patients of different shapes/sizes. Some patients may not adequately fit on the device, or need more elevation than offered by foam wedge 10. As a result, facilities that use a foam wedge often have to place extra pillows between the patient and the wedge to get the patient's hips high enough, and sometimes that still is not enough. Additionally, this method precludes the ability to accurately measure spinal opening pressure or to increase spinal pressure by injecting sterile saline into the spinal canal (to increase the pressure gradient towards the fistula), both of which require the patient in a horizontal position in order to be performed with accuracy and safety.

Therefore, other institutions, utilize inflatable mattress 14 shown in FIG. 2, which allows for patient 12 to begin the procedure in a horizontal position to allow for pressure measurement and manipulation, with subsequent inflation of mattress 14 to facilitate movement of contrast towards the head, followed by deflation of mattress 14 and the initiation of rapid image acquisition. While usually technically successful, it is suboptimal in terms of patient safety and reproducibility.

Accordingly, what is needed is an improved dynamic myelography positioning device configured to alter the orientation of a patient easily and quickly and adjust the height/angle of the device based on the dimensions and spinal characteristics of a specific patient. However, in view of the art considered as a whole at the time the present invention was made, it was not obvious to those of ordinary skill in the field of this invention how the shortcomings of the prior art could be overcome.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for an improved dynamic myelography positioning device is now met by a new, useful, and nonobvious invention.

The improved dynamic myelography positioning device includes a frame establishing a first end and a second end with a patient supporting structure integrated with or attachable to the frame. The device further includes a pivot mechanism residing between the first end and the second end of the frame. An actuator is in operable communication with at least a portion of the frame and is configured to change the height and slope of at least a portion of the frame.

Some embodiments include a pair of pivot mechanisms residing roughly halfway between the first end and the second end of the frame. The pivot mechanism may include a rotational axis that resides below a longitudinal axis of the frame when the frame is in a generally elongated, flat orientation. The pivot mechanism may also include one or more rotational stops configured to limit rotation to 180 degrees or less.

In some embodiments, the actuator is a winch actuator having a strap secured to the frame proximate the second end of the frame and the winch actuator is configured to reel in the strap thereby pulling the second end of the frame towards the first end of the frame. Some embodiments include a self-locking actuator.

Some embodiments include an actuator mount on which the actuator is secured and leveling feet on the underside of the actuator mount. Some embodiments further include one or more wheels secured to the second end of the frame. Moreover, in some embodiments, the patient supporting structure is comprised of non-metallic materials.

The present invention further includes a method of performing dynamic myelography. The method includes positing a patient on a dynamic myelography positioning device when the dynamic myelography positioning device resides on an imaging table; actuating an actuator thereby altering the slope of at least a portion of the dynamic myelography positioning device; injecting contrast into the patient; and acquiring one or more medical images configured to display CSF leaks.

Some embodiments further include measuring spinal opening pressure or increasing spinal pressure when the patient is in a generally horizontal position on the dynamic myelography positioning device.

Some embodiments of the method employ a dynamic myelography positioning device having a frame establishing a first end and a second end, a patient supporting structure integrated with or attachable to the frame wherein the patient supporting structure is comprised of non-metallic materials, a pivot mechanism residing between the first end and the second end of the frame, and the actuator in operable communication with at least a portion of the frame, such that the actuator is configured to cause at least one of the first end and the second end of the frame to move towards a respective other end of the frame.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 depicts a foam wedge method for patient elevation for dynamic myelography. The patient remains on the wedge for the duration of the procedure and spinal opening pressure cannot be calculated nor can pressure be manipulated; however, contrast material continues to move towards the head as scanning is performed, obviating the need to try to "time" the bolus.

FIG. 2 depicts an inflatable mattress method of patient elevation for dynamic myelography. Prior to inflation, access to the spinal canal is achieved with a needle, followed by pressure measurement and augmentation if necessary. The mattress is then inflated for a short period, deflated, and then rapid scanning initiated.

FIG. 3 is a perspective view of an embodiment of the present invention.

FIG. 4 is a perspective view of an embodiment of the frame of the present invention.

FIG. 5 is a close-up perspective view of a pair of pivot mechanisms attached to a pair of side frames.

FIG. 6 is a perspective view of an embodiment of the pivot mechanism.

FIG. 7 is a side view of the pivot mechanism in FIG. 6 attached to two side frames.

FIG. 8 is an elevation view of an embodiment of the present invention in a partially folded/rotated orientation.

FIG. 9 is a close-up top perspective view of an embodiment of the actuator secured to an end of the frame.

FIG. 10 is a close-up perspective view of an embodiment of the actuator secured to an end of the frame.

FIG. 11 is a perspective view of an embodiment of the actuator.

FIG. 12 is a close-up perspective view of an embodiment of the second end of the frame.

FIG. 13 is a close-up bottom perspective view of an embodiment of the second end of the frame.

FIG. 14 is a close-up perspective view of an embodiment of the second end of the frame.

FIG. 15 is a close-up top view of an embodiment of the present invention focusing on the strap attachment clamps.

FIG. 16 is a close-up top view of an embodiment of the strap attachment clamp secured to a side frame.

FIG. 17 is a close-up top view of an embodiment of the strap attachment clamp.

FIG. 18 is a side elevation view of an embodiment of the present invention in a partially folded/rotated orientation with a series of additional pillows.

FIG. 19 is a side elevation view of an embodiment of the present invention in a fully folded orientation.

FIG. 20 is a flowchart of an embodiment of the method of the present invention.

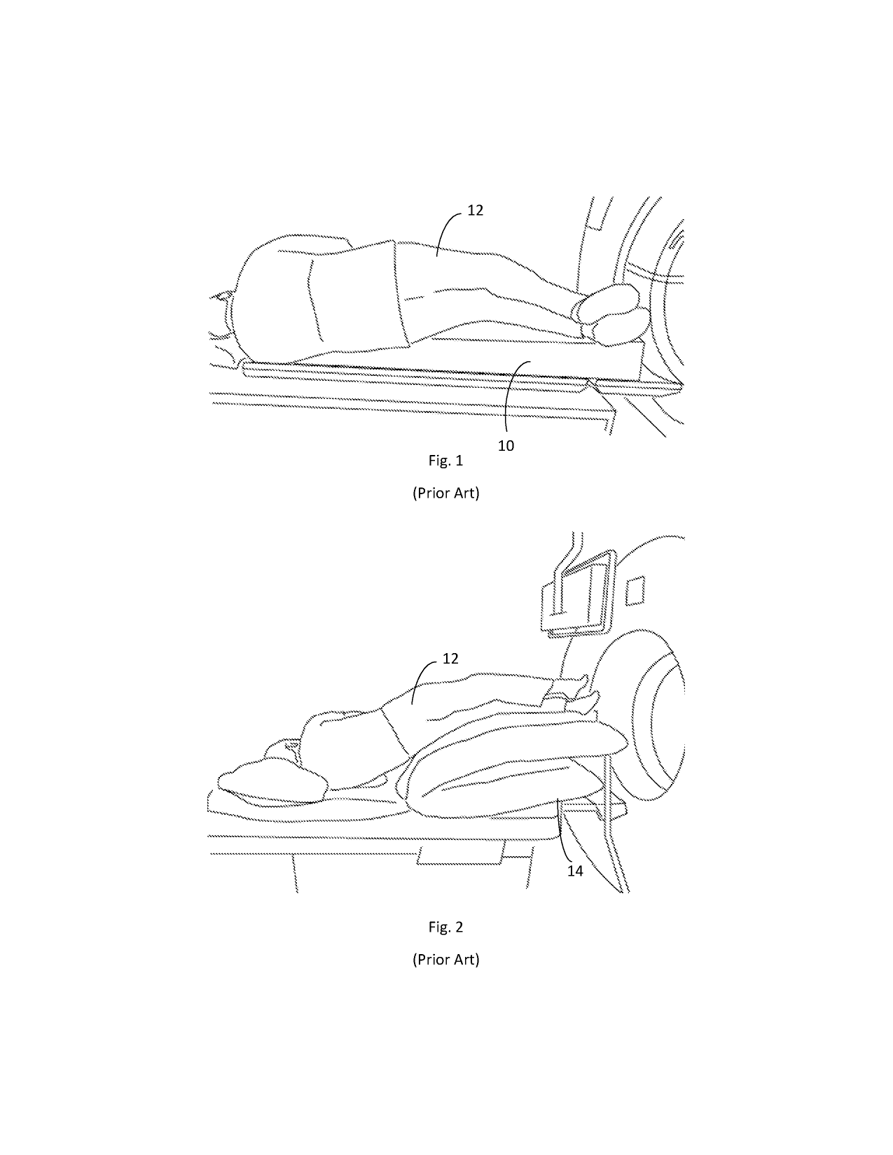

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms "a," "an," and "the" include plural referents unless the content clearly dictates otherwise. As used in this specification and the appended claims, the term "or" is generally employed in its sense including "and/or" unless the context clearly dictates otherwise.

The phrases "in some embodiments," "according to some embodiments," "in the embodiments shown," "in other embodiments," and the like generally mean the particular feature, structure, or characteristic following the phrase is included in at least one implementation. In addition, such phrases do not necessarily refer to the same embodiments or different embodiments.

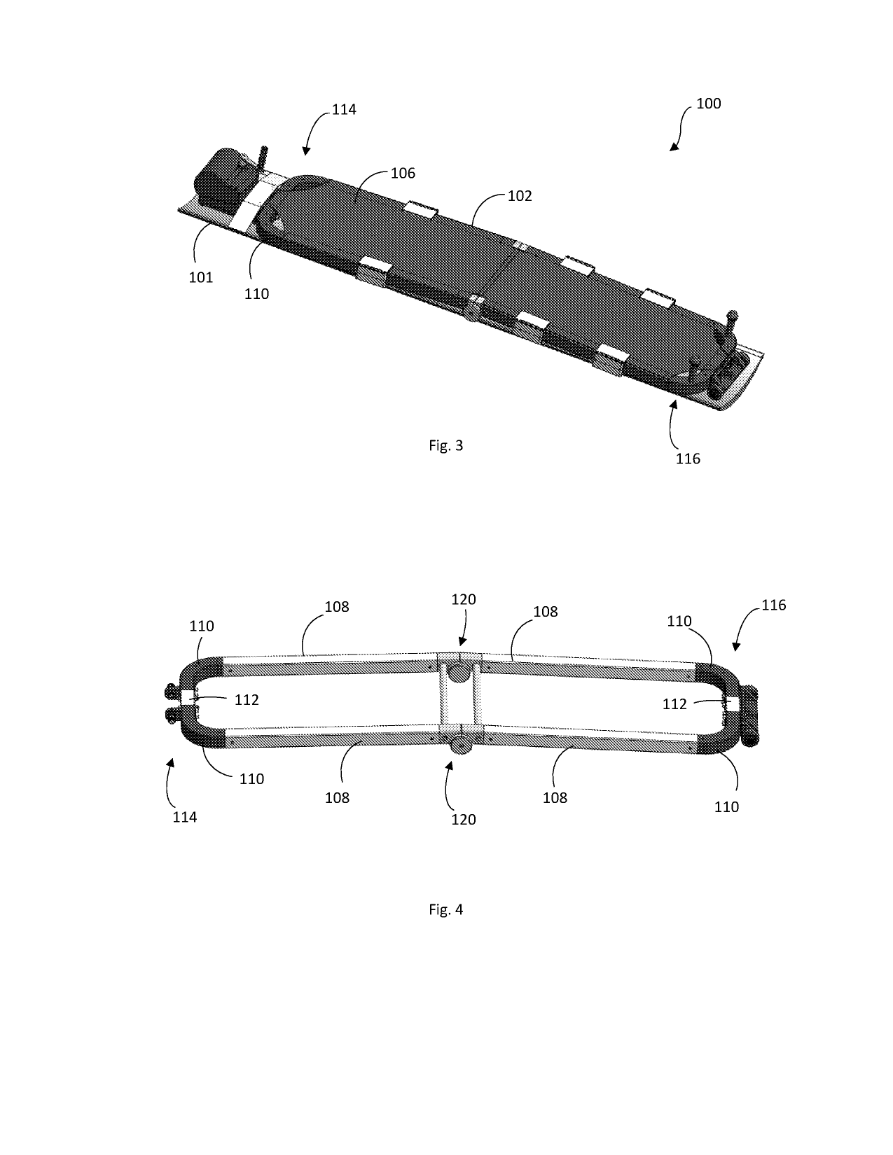

The present invention includes patient positioning device (PPD) 100 configured to move a patient from a generally flat laying position into a sloped or partially sloped position. In some embodiments, PPD 100 is attachable or configured to rest upon an existing imaging table 101 as shown in FIG. 3. However, in some embodiments, PPD 100 is integrated with or part of imaging table 101.

PPD 100 includes frame 102 with patient supporting structure 104 integrated with or attached to frame 102. Patient supporting structure 106 can be a high-strength fabric or any other material sufficient to support a patient. However, in some embodiments, patient supporting structure 106 is comprised of non-metallic material to minimize the patient's radioactive dosage during imaging.

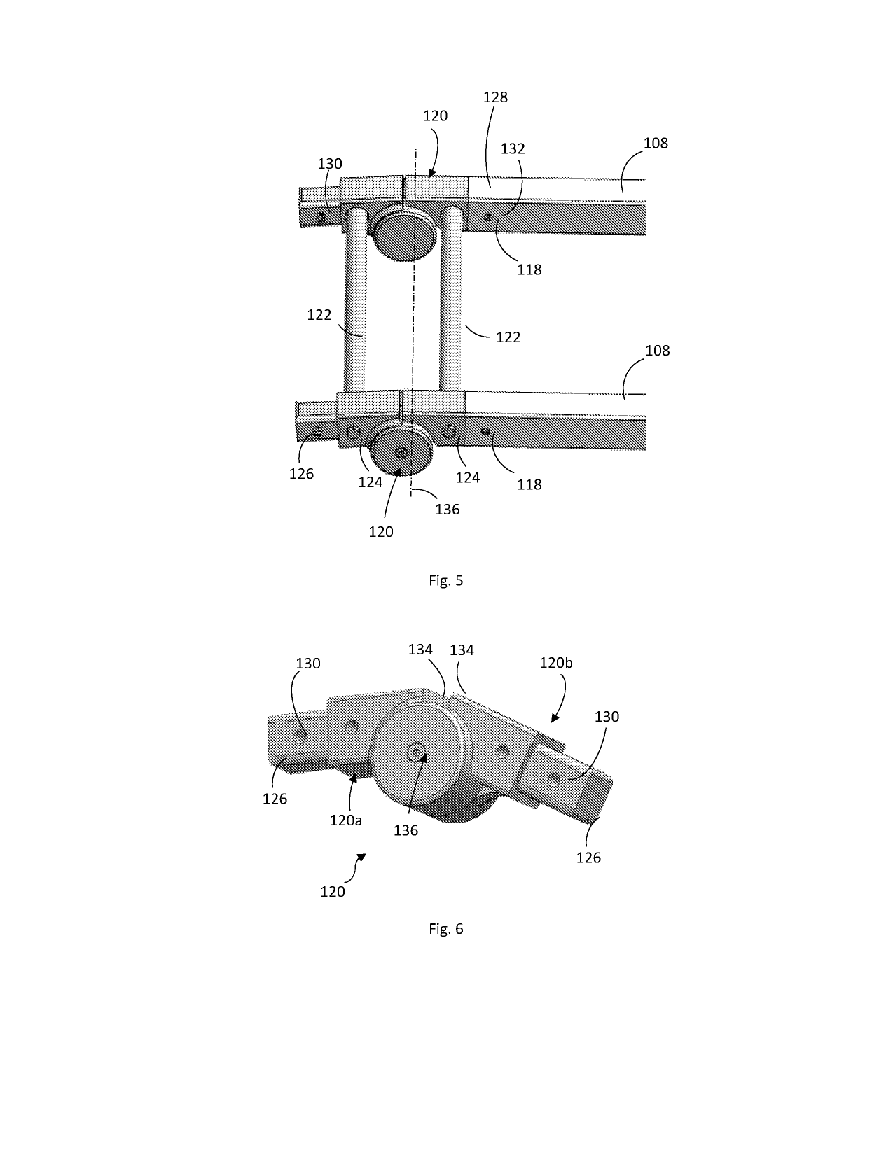

As best depicted in FIG. 4, frame 102 of PPD 100 is comprised of one or more members forming an elongated bed-shaped perimeter. The one or more members may include a multitude of side frames 108, corner brackets 110, and intermediate members 112. The one or more members can be interlocking through male extensions and female receipts. The various components can be secured using fasteners (e.g., fastener 118 in FIG. 5) or any other known devices and methods. As such, the various components may include fastener apertures (e.g., fastener apertures 132 in FIGS. 5-6) configured to aligned with each other.

Like patient supporting structure 106, in some embodiments, frame 102 of PPD 100, or at least a portion thereof, is comprised of non-metallic materials to reduce the patient's radioactive dosage during imaging. The portions of frame 102 that are likely to reside within the imaging window are the sections of frame 102 that can increase the patient's radioactive dosage during imaging. As such, these sections (e.g., the plurality of side frames 108) may be comprised of non-metallic materials to reduce the patient's radioactive dosage during imaging.

In the depicted embodiment, first end 114 and second end 116 each include a pair of corner brackets 110 with intermediate member 112 extending between each pair of corner brackets 110. However, it is contemplated that the end sections 114, 116 of frame 102 can comprise of a single member instead of a plurality of distinct members. In most instances, corner brackets 110 will not reside within the imaging window and thus, they can be comprised of stronger metallic materials to reinforce the strength of PPD 100 without increasing the radioactive exposure to the patient.

Each corner bracket 110 is integrated with or attachable to one of elongated side frames 108 that extends towards the opposing end of PPD 100. Corner brackets 110 can be secured to side frames 108 using fasteners 118 or any other known devices and methods. As such, corner brackets 110 may include fastener apertures aligned with similar apertures in side frames 108.

Some embodiments include a single side frame 108 on each side of PPD 100 that extends from a corner bracket 110 at first end 114 to a corner bracket 110 at second end 116. Some embodiments include multiple side frames 108 on each side of PPD 100. For example, the embodiment in FIG. 4 includes a pair of side frames 108 on each side of PPD 100 with pivot mechanism 120 residing between each pair of side frames 108.

In some embodiments, side frames 108 are adjustable in length. For example, each side frame 108 may be a telescoping assembly to adjust the total length of PPD 100 and/or the relative locations of the one or more pivot mechanisms 120 in relation to first end 114 and/or second end 116 of PPD 100. It should be understood that alternative approaches and mechanisms known in the art can be used to allow side frames 108 to adjust in length.

Some embodiments of PPD 100 include pivot mechanisms 120 residing between the first and second ends 114, 116 of PPD 100 to allow a user to alter the slope of at least a portion of PPD 100. Pivot mechanisms 120 may be provided at roughly the halfway point between the two ends 114, 116 of PPD 100 to allow a user to invert at least a portion of a patient's body, which is critical for certain imaging procedures as explained in the background section. Some embodiments include pivot mechanisms 120 located at multiple locations along the length of PPD 100 to account for individuals of different heights and allow for isolated inversion of portions of a patient's body. When multiple pivot mechanisms 120 are positioned at multiple locations along the length of PPD 100, said pivot mechanisms 120 can be lockable to isolate the pivotability of PPD 100 to specific rotational axes.

In some embodiments, a first pair of pivot mechanisms 120 is provided with one on each side of PPD 100 between adjacent side frames 108. In some embodiments, pivot mechanisms 120 are offset to the sides of PPD 100 and made of non-metallic materials to minimize radioactive exposure to the patient.

As best depicted in FIGS. 5, each pair of pivot mechanisms 120 can be structurally joined using one or more cross-members 122 connectable at attachment points on the mechanism. Cross-members 122 can be secured to pivot mechanisms 120 using fasteners 124 or other mechanisms and methods known in the art. Like other components in the imaging window, these cross-members 122 can be comprised of non-metallic materials to minimize radioactive exposure to the patient. In some embodiments, cross-members 122 can attach to side frames 108 rather than pivot mechanisms 120.

Each pivot mechanism 120 is also configured to be structurally joined to each of the adjacent side frames 108 through male extension 126 shaped and sized to be received by a corresponding female receipt 128 in an end of a side frame 108, or vice versa. In addition, each pivot mechanism 120 can be secured to a side frame 108 using fasteners 118 or other mechanisms and methods known in the art. Thus, pivot mechanisms 120 may include fastener apertures 130 configured to aligned with fastener apertures 132 of side frames 108.

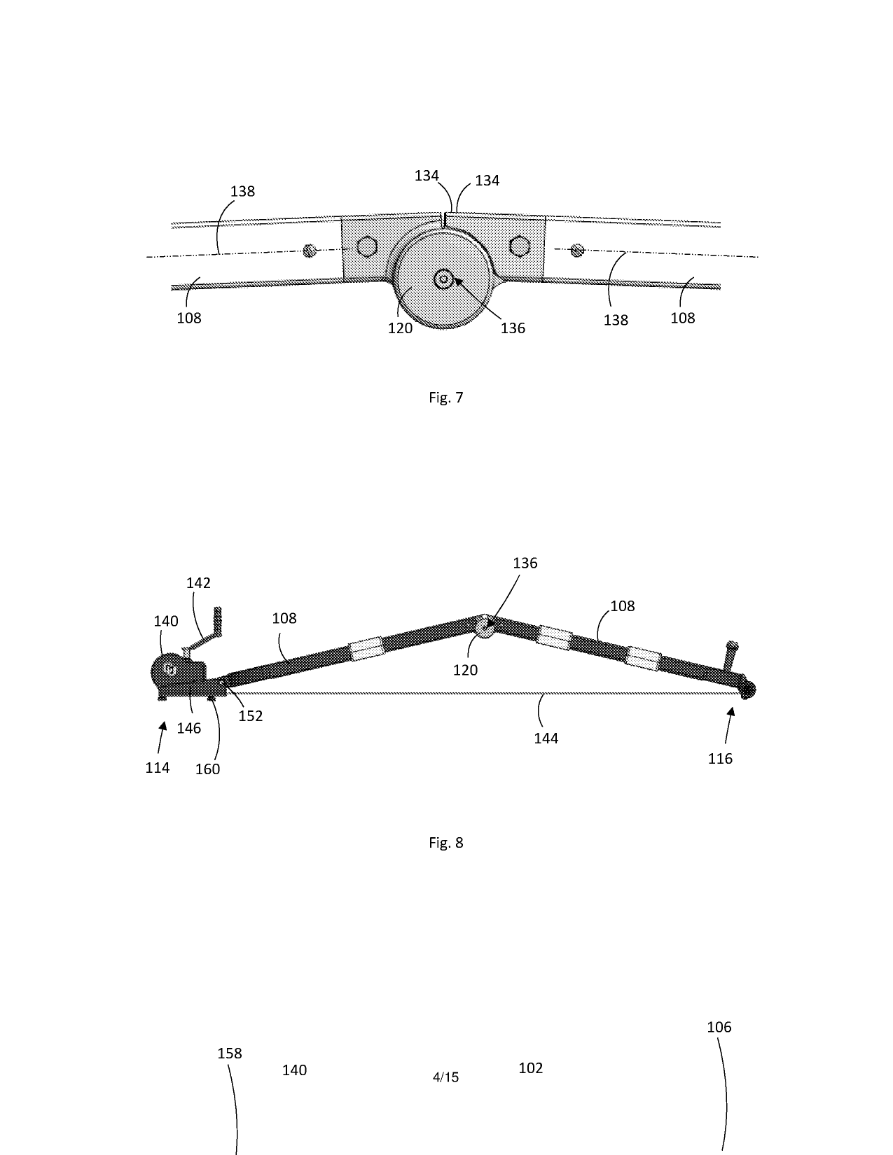

As best depicted in FIG. 6-7, each pivot mechanism 120 includes rotational stops 134 to restrict the direction and degree of rotation of the two rotating halves 120a, 120b of pivot mechanism 120. In some embodiments, rotational stops 134 prevent the adjoining side frames 108 from rotating beyond 180 degrees. In some embodiments, rotational stops 134 prevent the adjoining side frames 108 from reaching 180 degrees, which prevents PPD 100 from locking out in a completely flat orientation.

As best depicted in FIG. 7, rotational axes 136 of pivot mechanisms 120 are offset from

longitudinal axes 138 of side frames 108. Because rotational axes 136 are offset below longitudinal axes 138 of side frames 108, significantly less force is required to pivot PPD 100 about rotational axes 136 of pivot mechanisms 120. Some embodiments can also employ additional rotational assistance devices known in the art to aid in the initial rotation of PPD 100 out of the generally flat orientation. For example, pivot mechanism 120 may include spring mechanisms, such as torsion springs that add a rotational force on PPD 100 to help rotate PPD 100 out of the generally flat, non-rotated orientation.

Some embodiments also include one end of PPD 100 having a resting, non-rotated position in which it resides above the other end of PPD 100. If one end of PPD 100 is above the other end when PPD 100 is in a non-rotated orientation, less force is required to rotate the other end of PPD 100 about rotational axes 136 of pivot mechanisms 120. For similar reasons, some embodiments include a jack mechanism configured to increase the height of one side of PPD 100 prior to initiating the rotation of PPD 100 frame about pivot mechanism 120. When a jack mechanism is used, PPD 100 can have a completely flat resting, non-rotated orientation initially and then one end can be jacked up using the jack mechanism prior to initiating the rotation of PPD 100 frame about pivot mechanism 120 to substantially reduce the necessary input force to fold/rotate PPD 100.

In order to effect rotation/folding about rotational axes 136 of pivot mechanisms 120, the present invention includes actuator 140. In some embodiments actuator 140 is configured to draw at least one of the two ends 114, 116 of PPD 100 towards the other end by causing PPD 100 frame to fold or rotate about rotational axes 136 of pivot mechanisms 120.

In some embodiments, as best depicted in FIGS. 8-10, actuator 140 is a winch. The winch may be a manually powered winch (using e.g., hand crank 142) or a powered winch (using e.g., an electric motor). Actuator 140 is secured at one end of PPD 100 and retractable strap 144 is secured to the other end of PPD 100. Actuation of actuator 140 builds tension in retractable strap 144 eventually causing PPD 100 to rotate about rotational axes 136 of pivot mechanisms 120.

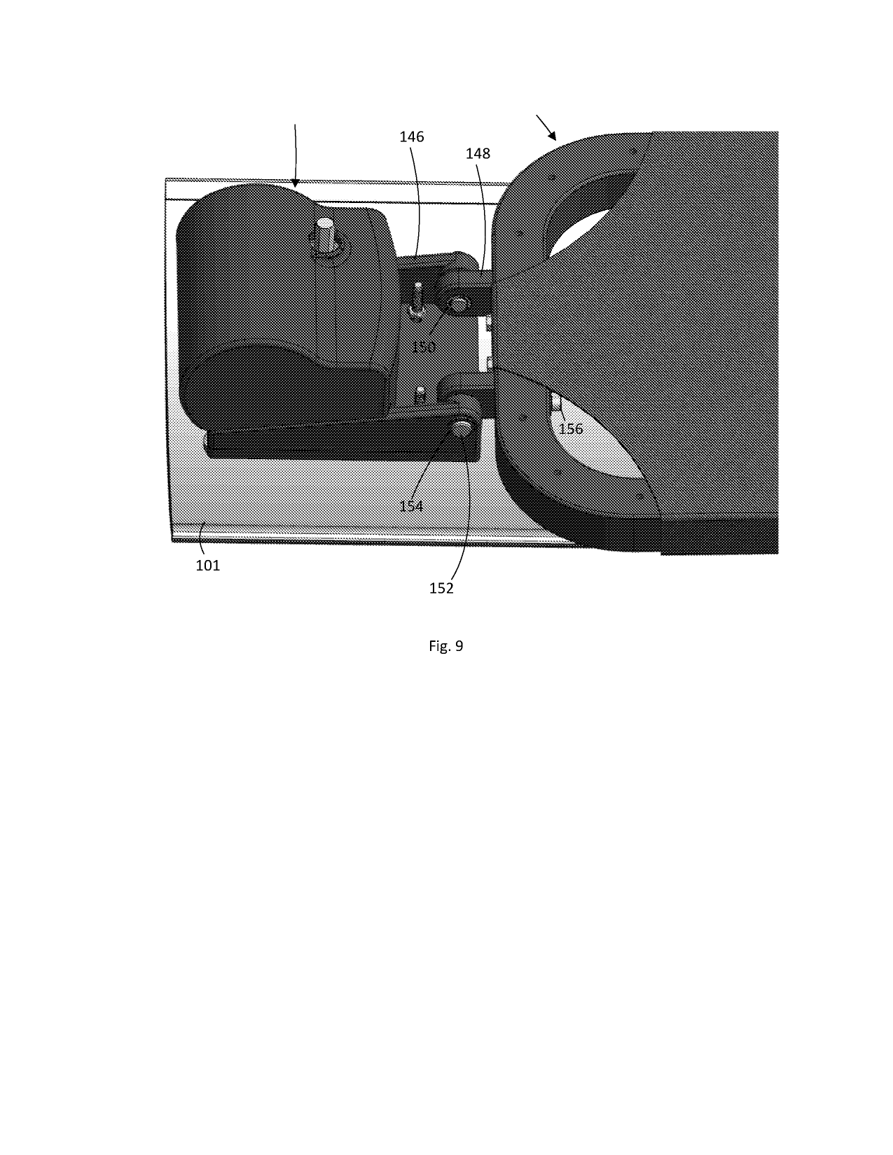

As exemplified in FIGS. 8-9, actuator 140 is rotatably secured to first end 114 of frame 102 of PPD 100 via actuator mount 146. As best depicted in FIG. 9, one or more structural extensions 148 project away from PPD 100 frame towards actuator 140. Extensions 148 include receipt 150 configured to receive rotational shaft 152. Actuator mount 146 also includes a similar receipt 154 configured to receive rotational shaft 152 such that PPD frame 102 is rotatably secured to actuator mount 146.

Structural extensions 146 can be secured to frame 102 using fasteners 156 or any other known devices and methods. As such, structural extensions 146 may include fastener apertures configured to align with apertures in frame 102. Moreover, while the depicted embodiment includes a rotational shaft passing through structural components connected to actuator mount 146 and frame extensions 148, alternative devices and methods known in the art may be used to rotatably secure actuator 140 (and/or actuator mount 146) to frame 102.

As depicted, actuator 140 is longitudinally offset away from patient supporting structure 106 to provide sufficient room for a user to actuate actuator 140 (e.g., rotate winch handle 142 when actuator 140 is a hand-crank winch) without contacting the patient. Actuator 140 is also preferably contained in protective housing 158 to prevent unintentional interaction between the moving components of actuator 140 and a patient.

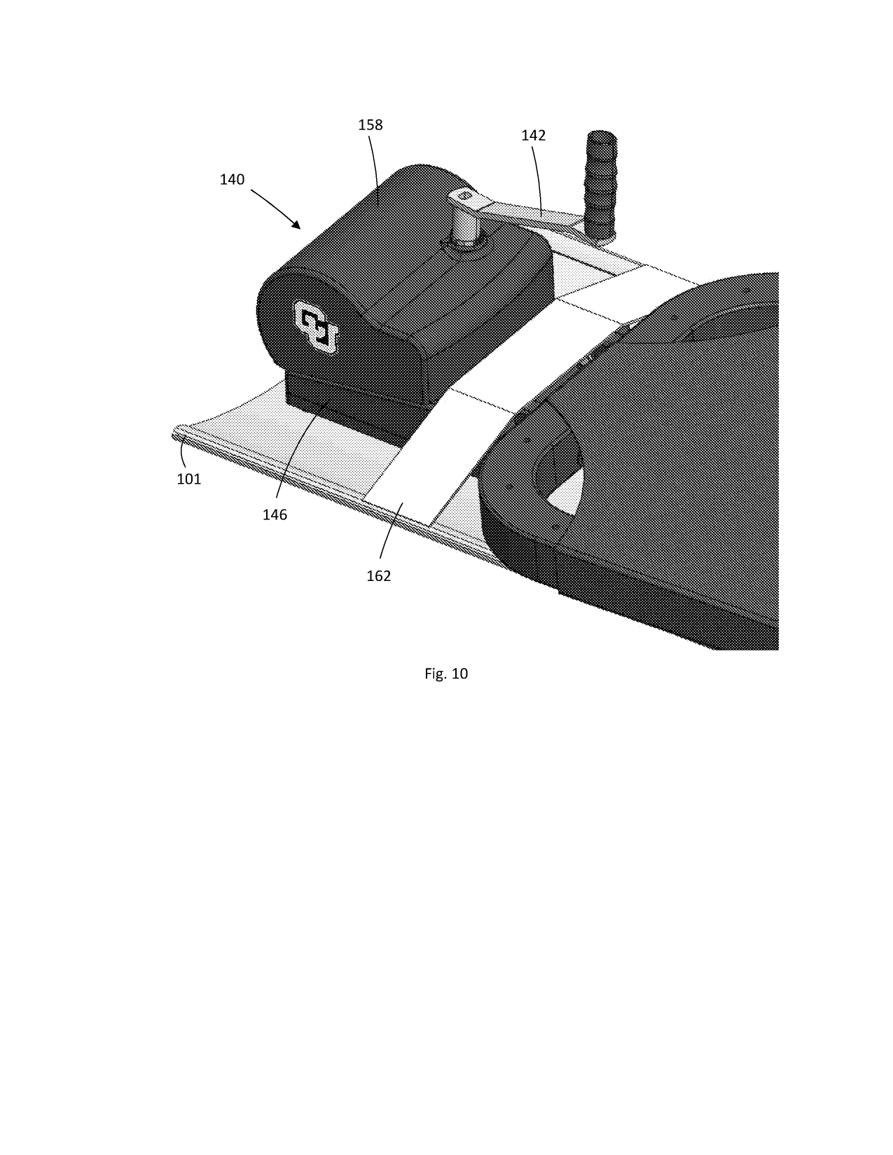

Actuator mount 146 may also include a series of adjustable leveling feet 160 (see FIG. 8) to level actuator 140 on the often-curved imaging tables 101. In some embodiments, as depicted in FIG. 10, PPD 100 includes actuator retention strap 162 to secure actuator 140 to imaging table 101. Actuator retention strap 162 can be secured to imaging table 101 via any known devices and methods known in the art. Alternatively, actuator mount 146 may be configured to connect to imaging table 101 without actuator retention strap 162.

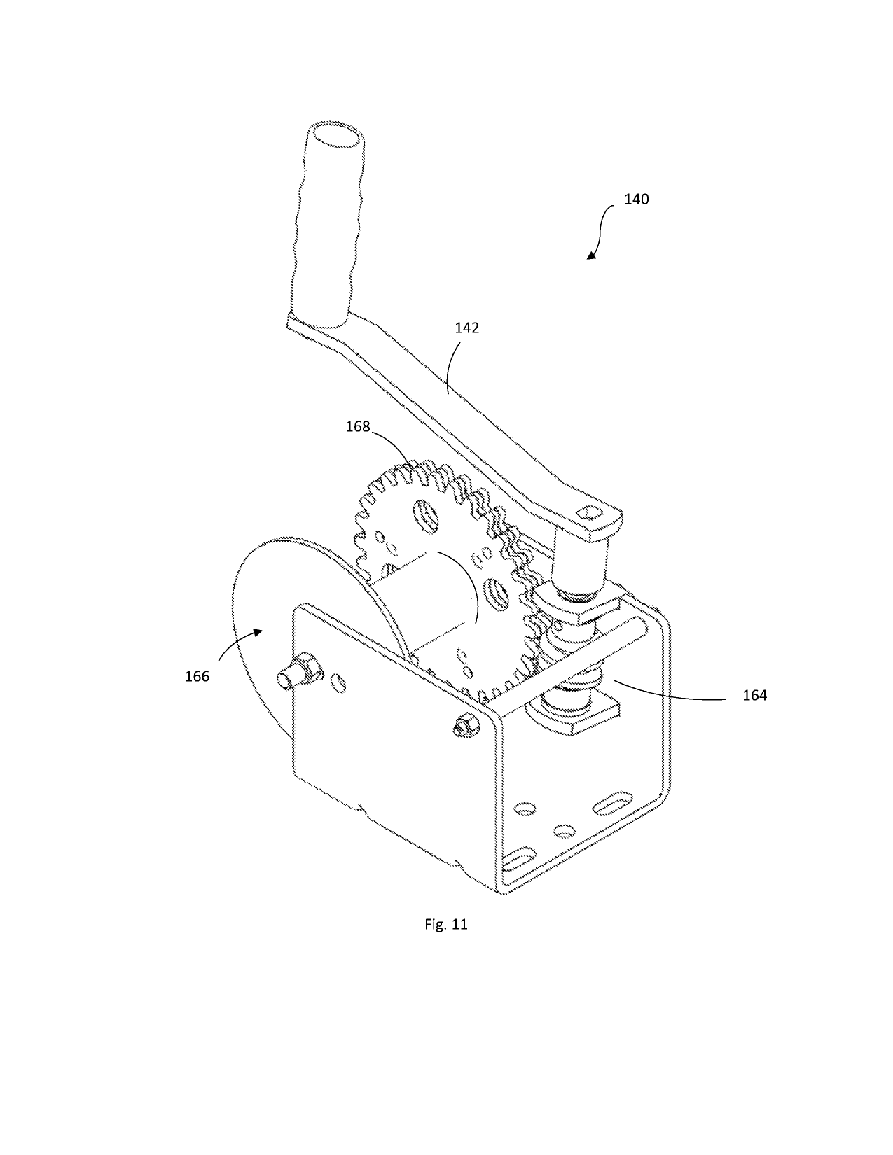

As best depicted in FIG. 11, some embodiments of actuator 140 include worm gear 164 to effect rotation of winch spool 166, through interconnecting teeth 168. A worm gear is an inherently self-locking gear, which prevents the patient's body weight from causing PPD 100 to reverse rotation and move back towards a generally flat orientation. For similar reasons, alternative self-locking gearing or actuators may be used to effect rotation of winch spool 166.

Some embodiments may use alternative actuators to effect rotation of PPD 100 about one or more of the rotational axes or to alter the height and slope of at least a portion of PPD 100. Non-limiting examples include pneumatic pillows, pistons, jacks, and linear actuators.

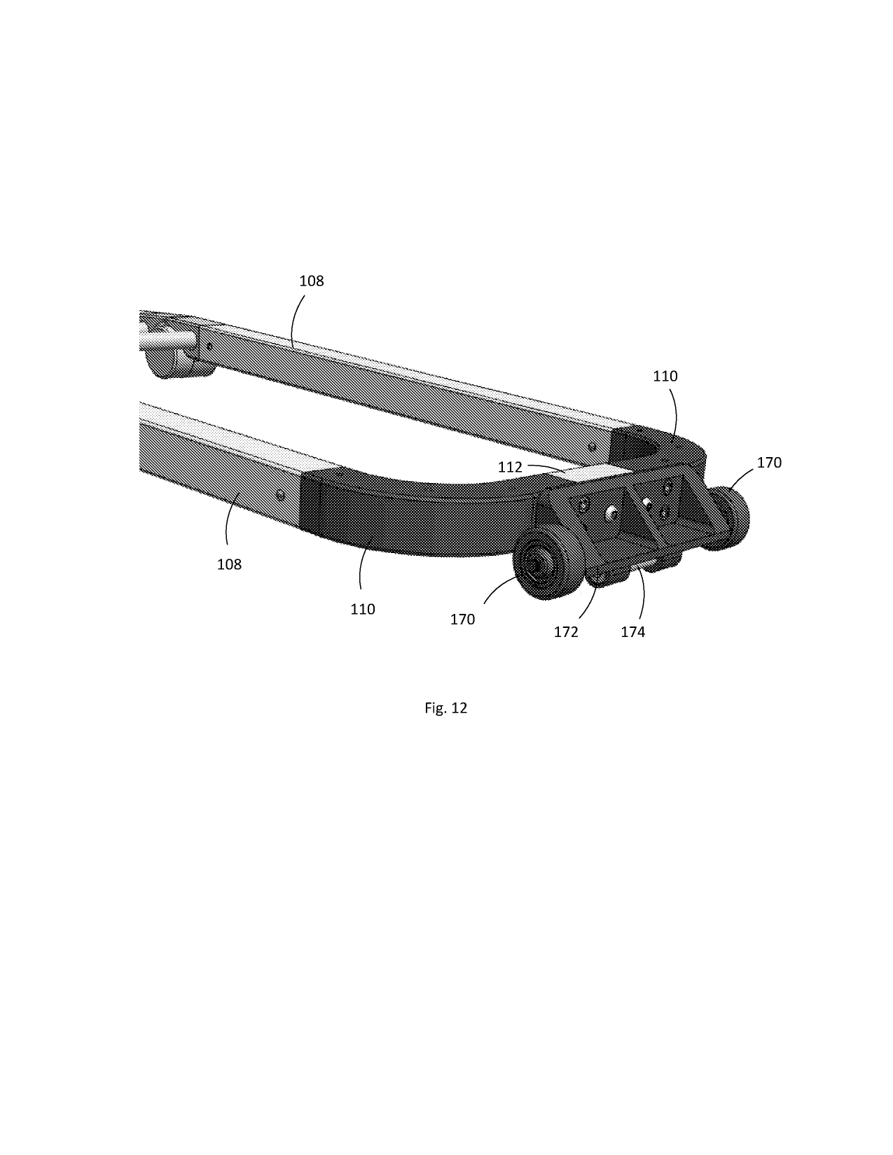

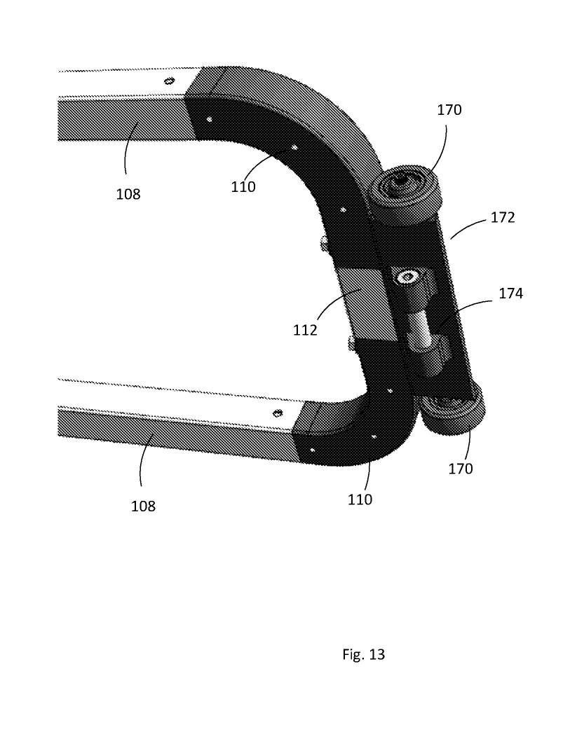

Referring now to FIGS. 12-14, PPD 100 can include one or more wheels 170 on the end opposite from actuator 140. The one or more wheels 170 are preferably non-marking wheels and comprised of non-metallic materials, e.g., rubber.

The depicted embodiment includes a pair of wheels 170 secured to second end 116 of PPD 100. Wheels 170 reduce the friction between imaging table 101 and PPD 100 as second end 116 is pulled towards first end 114 of PPD 100. To ensure that wheels 170 contact imaging table 101 instead of frame 102, wheels 170 are offset towards the underside of frame 102 or have a sufficiently large diameter such that when PPD 100 is in the generally flat orientation, second end 116 of frame 102 is elevated above imaging table 101 when wheels 170 are resting on imaging table 101.

Some embodiments of PPD 100 include wheel mount 172 that is attachable to at least a portion of frame 102, e.g., intermediate member 112 and/or corner brackets 110. Wheel mount 172 can be secured to frame 102 using fasteners or any other known devices and methods. As such, wheel mount 172 may include fastener apertures aligned with similar apertures in frame 102.

Wheel mount 172 may further include strap mount 174 when actuator 140 is a winch actuator. Strap mount 174 can be secured to the underside of wheel mount 172 or may be secured to one of the frame members at a location opposite the winch actuator. Strap mount 174 extends downwardly towards imaging table 101 and includes a strap receiving section. The downward extent is sufficiently small to ensure that it does not touch imaging table 101 when PPD 100 is in the generally flat orientation with wheels 170 resting on imaging table 101.

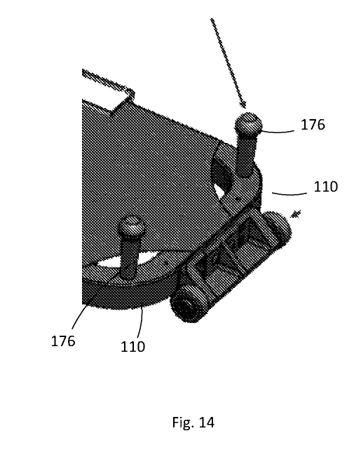

As best shown in FIG. 14, PPD 100 can further include a pair of handles 176 secured at one or both ends of PPD 100. Handles 176 provide a patient with a means for supporting themselves and increasing comfort. In some embodiments, handles 176 include a threaded male extension configured to pass through one or more apertures in corner brackets 110. The receiving apertures can be threaded or the male extension is sized to pass completely through corner brackets 110 and a nut is used to secure handles 176 in place. However, alternative devices and methods known in the art can be used to secure handles 176 to PPD frame 102.

In some embodiments, corner brackets 110 on each end of PPD 100 are identical. As such, handles 176, wheel mount 172, and actuator mount 146 can attach to either end of PPD frame 102 as needed.

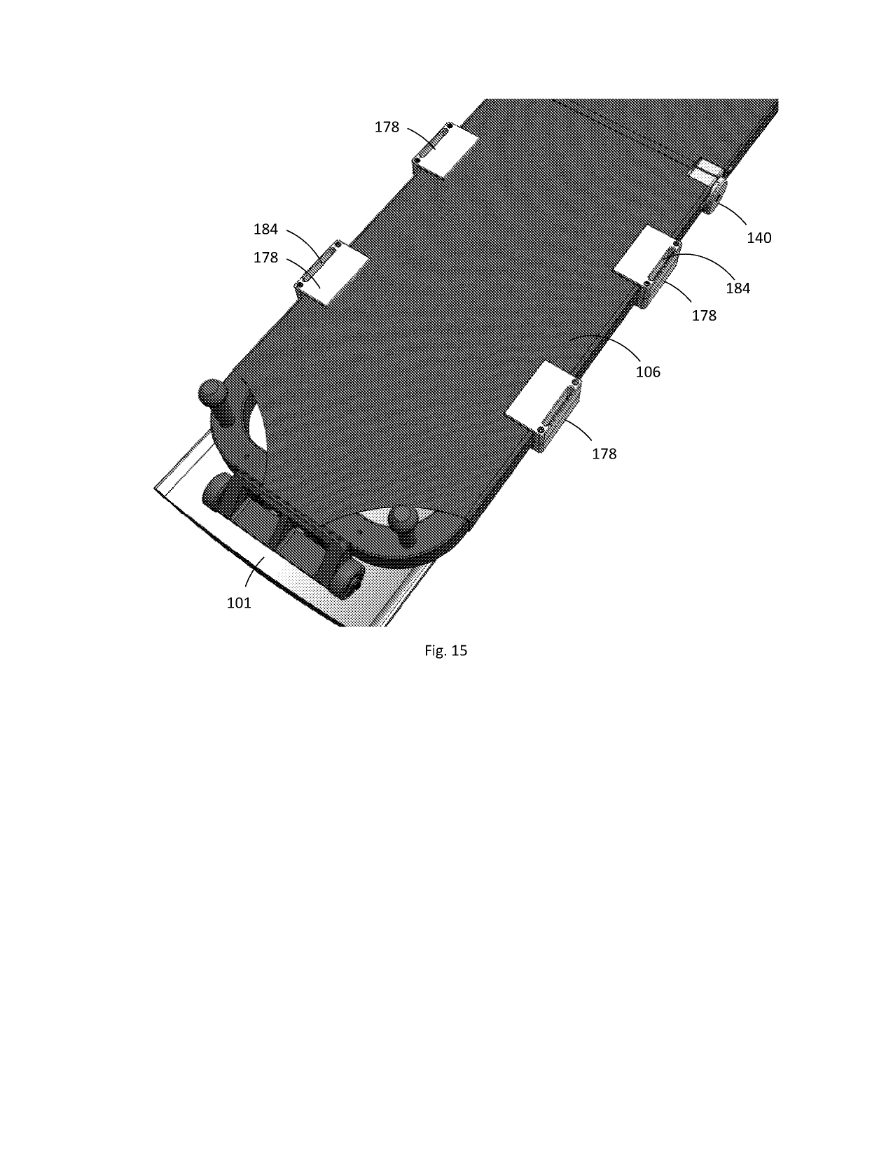

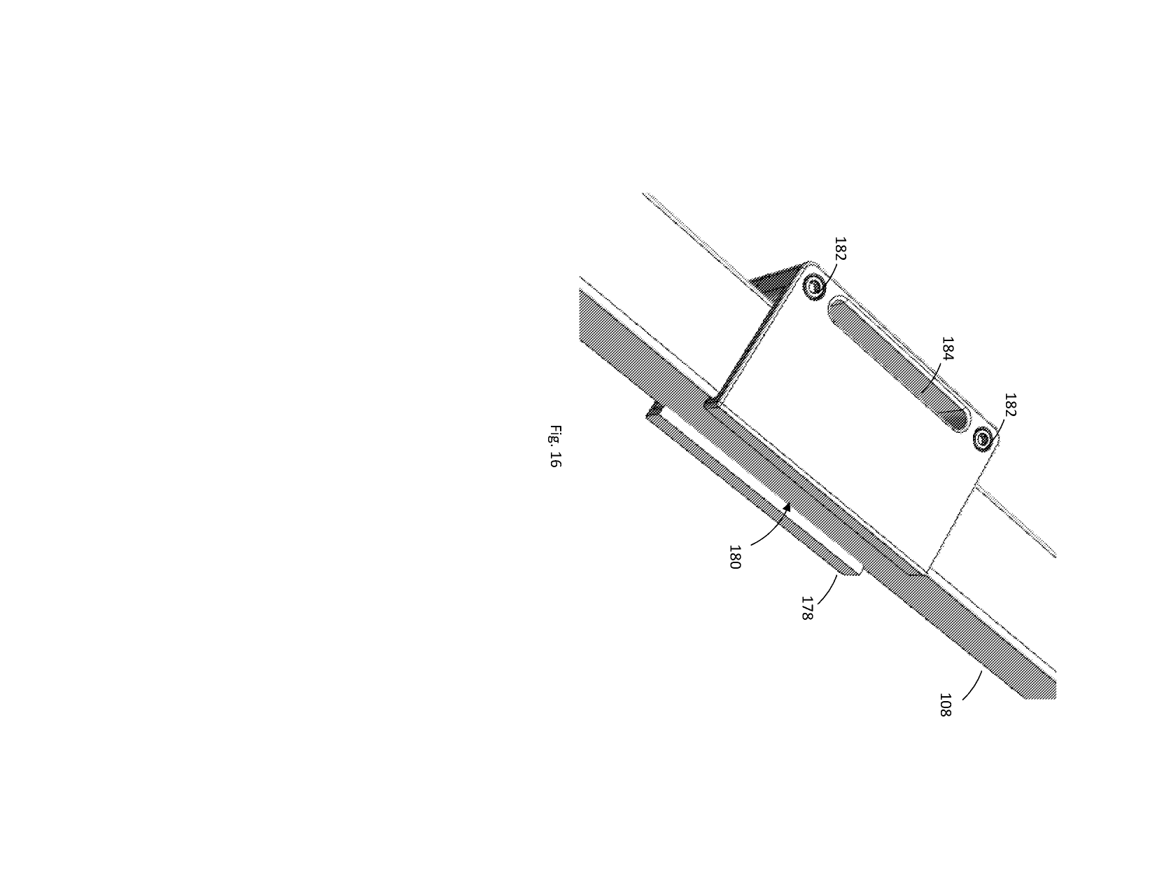

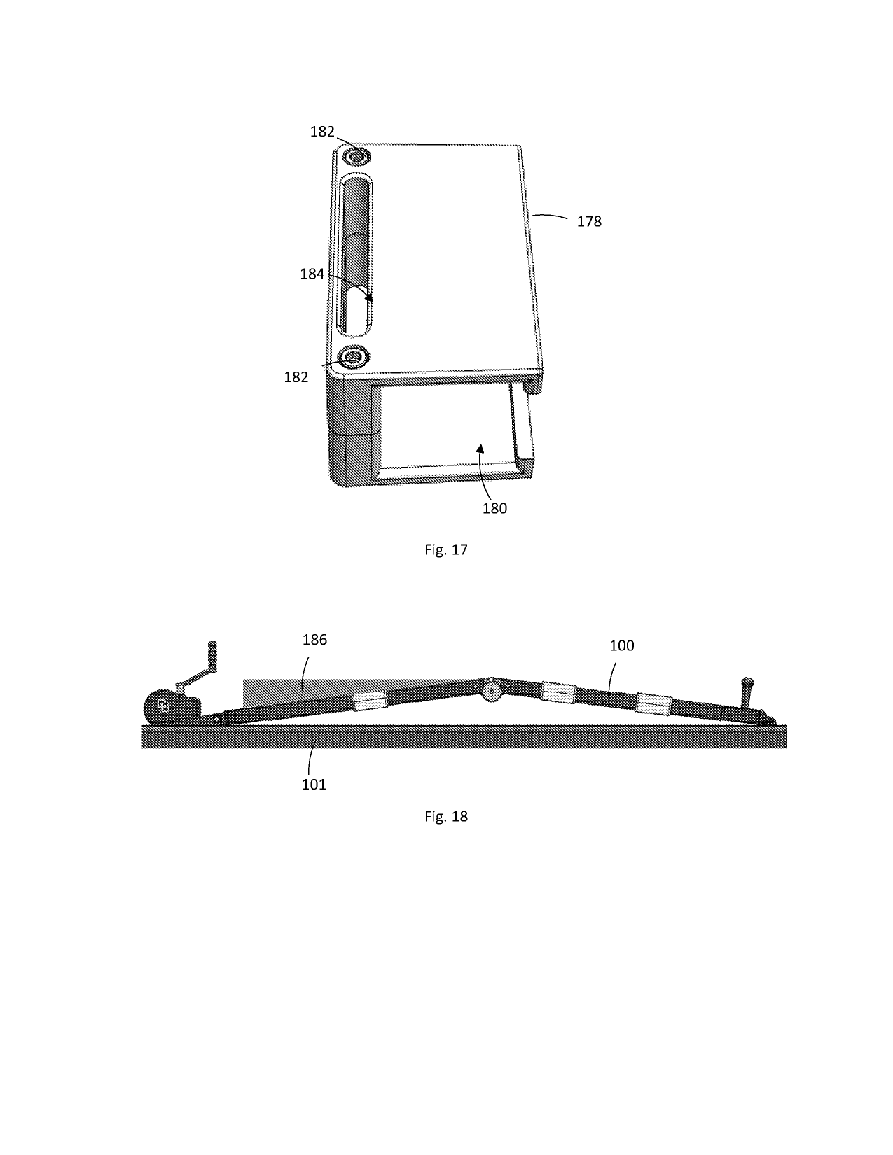

Referring now to FIGS. 15-17, some embodiments include a series of slidable strap attachment clamps 178. Each clamp 178 is sized and shaped to receive a portion of frame 102, e.g., side frame 108, within passageway 180 defined by the body of clamp 178. The body wraps around frame 102 in a manner to secure clamp 178 to frame 102. In some embodiments, each clamp 178 is a multi-part construction that can be adjusted to allow each clamp 178 to slide along the length of frame 102 and tightened to prevent sliding along frame 102. The adjustability can be achieved through fasteners 182 as provided in the exemplary figures or through other known devices and methods.

Each clamp 178 further includes strap receipt 184 configured to receive a patient- retention strap. The exemplary receipt 184 depicted is a through-hole. However, alternative approaches can be used to secure the patient-retention straps. In some embodiments, the slidable strap attachment clamps 178 and straps are comprised of non- metallic materials to reduce the patient's radioactive dosage.

As exemplified in FIG. 18, some embodiments of the present invention include one or more wedges, pads, or pillows (collectively referred to as "inserts 186") that can be used with PPD 100. Inserts 186 can be used to alter the position of the patient in addition to the adjustability of PPD 100. In addition, inserts 186 are preferably sized to reside between pivot mechanism 120 and an end of PPD 100. Similar to other components, inserts 180 are comprised of non-metallic materials to reduce the patient's exposure to radiation.

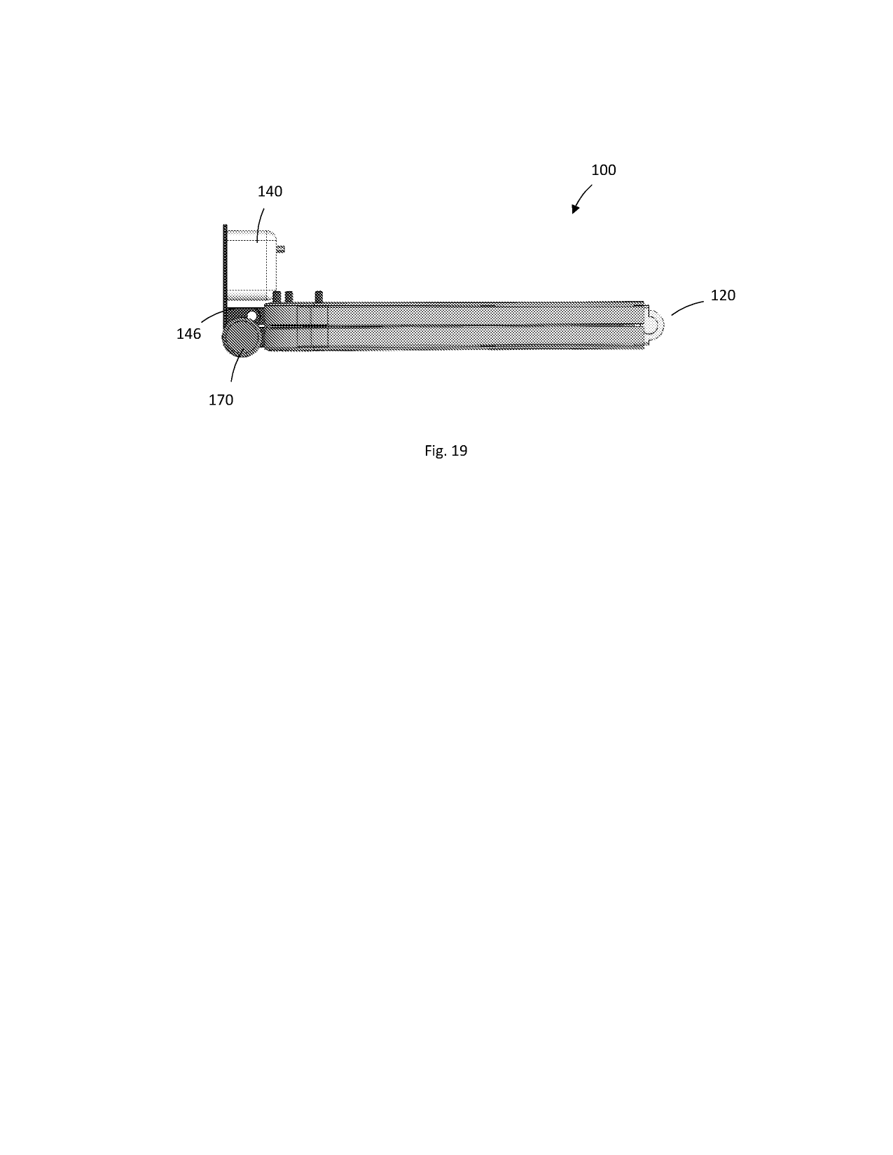

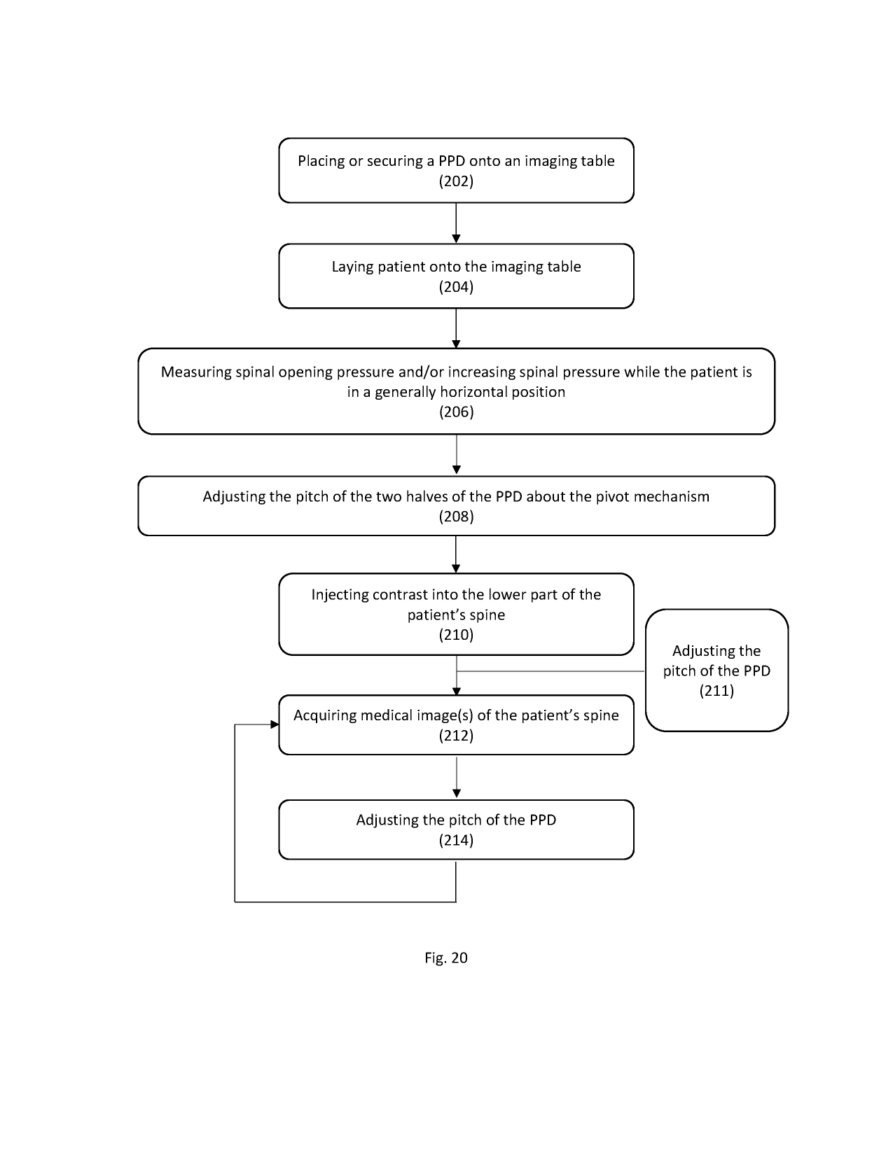

Referring now to FIG. 19, some embodiments of PPD 100 are configured to fold about pivot mechanism 120 until the two ends reside generally next to each other. Actuator mount 146 can also fold upwardly to reduce the overall length of PPD 100. The device can then fit into a carrying bag for storage and transportation.

The present invention further includes a method of performing dynamic myelography using a PPD, such as one of the various embodiments of PPD 100 described above. The method includes placing or securing the PPD onto an imaging table at step 202 and the patient laying on the table at step 204. Some embodiments of the method include step 206 which includes measuring spinal opening pressure and/or increasing spinal pressure by injecting sterile saline into the spinal canal while the patient is in a generally horizontal position. After step 204 or after optional step 206, the PPD is then adjusted to vary the pitch of the two halves of the PPD about the pivot mechanism at step 208, which is exemplified in FIG. 8. Adjusting the pitch includes actuating the actuator, which may be configured to move one or more of the ends of the PPD relative to each other.

Once the patient is in the correct orientation, contrast is injected into the lower part of the patient's spine at step 210 and gravity moves the contrast towards the top of the patient's spine. Alternatively, the injection of contrast can occur while the patient is in a generally flat orientation and orientation of the PPD can be adjusted following the injection of the contrast.

One or more medical images (e.g., x-ray or CT images) of the patient are then acquired at step 212 to identify the locations of CSF leaks. Some embodiments include additional steps 211 and/or 214. Both steps 211 and 214 include adjusting the pitch of the PPD to change the orientation of the patient (including returning to a generally horizontal orientation) in an attempt to control the flow of the injected contrast. When step 214 is performed, step 212 is performed after step 214 or both before and after step 214 as needed to identify the locations of CSF leaks.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A dynamic myelography positioning device, comprising:
a frame establishing a first end and a second end;
a patient supporting structure integrated with or attachable to the frame;
a pivot mechanism residing between the first end and the second end of the frame; and
an actuator in operable communication with at least a portion of the frame, wherein the actuator is configured to change the slope and height of at least a portion of the frame.

2. The dynamic myelography positioning device of claim 1, further including a pair of pivot mechanisms residing roughly halfway between the first end and the second end of the frame.

3. The dynamic myelography positioning device of claim 1, wherein the pivot mechanism includes a rotational axis that resides below a longitudinal axis of the frame when the frame is in a generally elongated, flat orientation.

4. The dynamic myelography positioning device of claim 1, wherein the pivot mechanism includes one or more rotational stops configured to limit rotation to 180 degrees or less.

5. The dynamic myelography positioning device of claim 1, wherein the actuator is a winch actuator having a strap secured to the frame proximate the second end of the frame and the winch actuator is configured to reel in the strap thereby pulling the second end of the frame towards the first end of the frame.

6. The dynamic myelography positioning device of claim 1, wherein the actuator is self-locking.

7. The dynamic myelography positioning device of claim 1, further including an actuator mount on which the actuator is secured and leveling feet on the underside of the actuator mount.

8. The dynamic myelography positioning device of claim 1, further including one or more wheels secured to the second end of the frame.

9. The dynamic myelography positioning device of claim 1, wherein the patient supporting structure is comprised of non-metallic materials.

10. A dynamic myelography positioning device, comprising:
a frame establishing a first end and a second end;
a patient supporting structure integrated with or attachable to the frame wherein the patient supporting structure is comprised of non-metallic materials;
a pivot mechanism residing between the first end and the second end of the frame; and
an actuator in operable communication with at least a portion of the frame, wherein the actuator is configured to cause at least one of the first end and the second end of the frame to move towards a respective other end of the frame.

11. The dynamic myelography positioning device of claim 10, further including a pair of pivot mechanisms residing roughly halfway between the first end and the second end of the frame.

12. The dynamic myelography positioning device of claim 10, wherein the pivot mechanism includes a rotational axis that resides below a longitudinal axis of the frame when the frame is in a generally elongated, flat orientation.

13. The dynamic myelography positioning device of claim 10, wherein the pivot mechanism includes one or more rotational stops configured to limit rotation to 180 degrees or less.

14. The dynamic myelography positioning device of claim 10, wherein the actuator is a winch actuator having a strap secured to the frame proximate the second end of the frame and the winch actuator is configured to reel in the strap thereby pulling the second end of the frame towards the first end of the frame.

15. The dynamic myelography positioning device of claim 10, wherein the actuator is self-locking.

16. The dynamic myelography positioning device of claim 10, further including an actuator mount on which the actuator is secured and leveling feet on the underside of the actuator mount.

17. The dynamic myelography positioning device of claim 10, further including one or more wheels secured to the second end of the frame.

18. A method of performing dynamic myelography, comprising:
positing a patient on a dynamic myelography positioning device, wherein the dynamic myelography positioning device resides on an imaging table;
actuating an actuator thereby altering the slope and height of at least a portion of the dynamic myelography positioning device;
injecting contrast into the patient; and
acquiring one or more medical images, wherein the medical images are configured to display cerebrospinal fluid leaks.

19. The method of claim 18, further including measuring spinal opening pressure or increasing spinal pressure when the patient is in a generally horizontal position on the dynamic myelography positioning device.

20. The method of claim 18, wherein the dynamic myelography positioning device includes:
a frame establishing a first end and a second end;
a patient supporting structure integrated with or attachable to the frame wherein the patient supporting structure is comprised of non-metallic materials;
a pivot mechanism residing between the first end and the second end of the frame; and
the actuator in operable communication with at least a portion of the frame, wherein the actuator is configured to cause at least one of the first end and the second end of the frame to move towards a respective other end of the frame.
